(19) United States
(12) Patent Application Publication
(10) Pub. No.: US 2008/0182899 A1
(43) Pub. Date: Jul. 31, 2008

(54) ASSOCIATION OF BETA3 RECEPTOR AGONIST AND MONOAMINE REUPTAKE INHIBITORS, PHARMACEUTICAL COMPOSITION AND THERAPEUTIC USE THEREOF

(75) Inventors: Guy GRIEBEL, Fontenay Aux Roses (FR); Jeanne STEMMELIN, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/050,557

(22) Filed: Mar. 18, 2008

Related U.S. Application Data
(63) Continuation of application No. PCT/FR2006/002125, filed on Sep. 18, 2006.

(30) Foreign Application Priority Data
Sep. 19, 2005 (FR) 0509528

(51) Int. Cl. A61K 31/24; A61K 31/195

(52) U.S. Cl. 514/534; 514/567

(57) ABSTRACT

The invention concerns the association of at least one β3 adrenergic receptor agonist (or β3 agonist) with a monoamine reuptake inhibitor. The invention also concerns a pharmaceutical composition comprising the inventive association and its therapeutic use.

This application is a continuation of International application No. PCT/FR2006/002,125, filed Sep. 18, 2006, which is incorporated herein by reference in its entirety; which claims the benefit of priority of French Patent Application No. 05/09,528, filed Sep. 19, 2005.

The invention relates to a combination of at least one β3 adrenergic receptor agonist (or β3 agonist) with a monoamine reuptake inhibitor (MARI).

The invention also relates to a pharmaceutical composition comprising the combination of the invention and to the therapeutic use thereof.

Phenylethanolaminotetralins are known as lipolytic and intestinal spasmolytic agents acting via a mechanism of selective simulation of atypical β receptors (neither β1 nor β2) present in the intestine. Their preparation and characterization have been described in particular in documents EP-A-211 721, EP-A-303 545, EP-A-303 546 and EP-A-383 686.

The use of β3 adrenergic receptor agonists such as phenylethanolaminotetralins as antidepressants has also been described in document EP-A-489 640.

Among the monoamine reuptake inhibitors, serotonin and noradrenalin reuptake inhibitors are known as antidepressants and are described especially in document EP-A-958 824.

There is still a need for medicaments that are even more efficient for treating patients suffering from depression or anxiety.

The invention is directed towards satisfying this aim, by proposing a combination of a β3 receptor agonist and a monoamine reuptake inhibitor, this combination having improved action compared with the action of the two active principles taken individually.

A first subject of the invention thus concerns such a combination.

A second subject of the invention concerns a pharmaceutical composition comprising, as active principle, such a combination.

A third subject of the invention relates to the use of such a combination for the treatment and/or prevention of depression or anxiety.

Thus, a first aspect of the invention relates to the combination of at least one β3 adrenergic receptor agonist with at least one MARI.

For the purposes of the present invention, the general term “MARI” means monoamine reuptake inhibitors. Examples that may especially be mentioned include selective serotonin reuptake inhibitors (SSRI), selective noradrenalin reuptake inhibitors (SNRI), dopamine reuptake inhibitors, dopamine and noradrenalin reuptake inhibitors, and also serotonin and noradrenalin reuptake inhibitors.

According to a first embodiment of the invention, the β3 adrenergic receptor agonist is a phenylethanolaminotetralin of general formula (I):

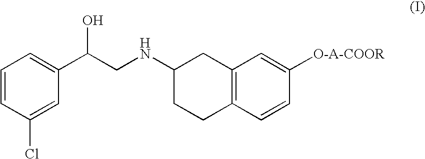

in which:
A represents a C₁₋₄ alkylene group, and
R represents a hydrogen atom or a C₁₋₄ alkyl group, in the form of base or of acid-addition salt.

The salts that may be used may be prepared with pharmaceutically acceptable acids, but the salts of other acids that are useful, for example, for the purification or isolation of the phenylethanolaminotetralins of formula (I), also form part of the invention.

The phenylethanolaminotetralins of general formula (I) may exist in the form of hydrates or solvates, i.e. in the form of associations or combinations with one or more water molecules or with a solvent. Such hydrates and solvates also form part of the invention.

The phenylethanolaminotetralins of general formula (I) may comprise one or more asymmetric carbon atoms. They may thus exist in the form of enantiomers or diastereoisomers.

These enantiomers and diastereoisomers, and also mixtures thereof, including racemic mixtures, form part of the invention.

In the context of the present invention, the following definitions apply:
C_{t-z} in which t and z may take values from 1 to 4, a carbon-based chain possibly containing from t to z carbon atoms, for example C₁₋₄ a carbon-based chain possibly containing from 1 to 4 carbon atoms;
an alkylene group: a linear, branched or cyclic, saturated divalent alkyl group, for example a C₁₋₄-alkylene group represents a linear or branched divalent carbon-based chain of 1 to 4 carbon atoms, for example a methylene, ethylene, 1-methylethylene, propylene, isopropylidene or butylene;
an alkyl group: a linear, branched or cyclic, saturated aliphatic group; for example a C₁₋₄-alkyl group represents a carbon-based chain possibly containing from 1 to 4 carbon atoms, for example a methyl, ethyl, propyl, isopropyl, butyl, isobutyl or tert-butyl.

A first group of phenylethanolaminotetralins that may be used according to the invention is that of formula (I) in which:

A represents a methylene or isopropylidene group and R represents a hydrogen atom or a C₁₋₄ alkyl group.

Among the phenylethanolaminotetralins that may be used in the context of the invention, mention may be made especially of ethyl [2-(3-chlorophenyl)-2-hydroxyethyl-amino-5,6,7,8-tetrahydronaphthalen-2-yloxy]acetate.

Among the diastereoisomers of ethyl [2-(3-chlorophenyl)-2-hydroxyethylamino-5,6,7,8-tetrahydronaphthalen-2-yloxy]-acetate, mention may be made especially of ethyl [(7S)-7(2R)-2-(3-chlorophenyl)-2-hydroxyethylamino-5,6,7,8-tetra-hydronaphthalen-2-yloxy]acetate.

Examples of phenylethanolaminotetralin salts that may be used according to the invention are ethyl [2-(3-chloro-phenyl)-2-hydroxyethylamino-5,6,7,8-tetrahydronaphthalen-2-yloxy]acetate hydrochloride and ethyl [(7S)-7(2R)-2-(3-chlorophenyl)-2-hydroxyethylamino-5,6,7,8-tetrahydro-naphthalen-2-yloxy]acetate hydrochloride.

The above compounds are described especially in document EP-A-303 546.

Ethyl [(7S)-7(2R)-2-(3-chlorophenyl)-2-hydroxyethylamino-5,6,7,8-tetrahydronaphthalen-2-yloxy]acetate hydrochloride may also be in a particular crystalline form, known as the B form (also known as form 2). The B form, which is described in document EP-A-1 404 641 (also see U.S. Pat. No. 6,992,211), especially has the following characteristics:
characteristic IR absorption peaks (cm⁻¹): 2780, 2736, 1722, 1211;
melting point: 129±2° C.;
characteristic lines in the powder X-ray diffraction diagram (to within 0.1(2θ)): 7.69-9.83-13.95-16.58-18.70-20.40-21.57-23.40-24.15-25.64.

The B form of ethyl [(7S)-7(2R)-2-(3-chlorophenyl)-2-hydroxyethylamino-5,6,7,8-tetrahydronaphthalen-2-yloxy]-acetate hydrochloride generally corresponds to a product comprising at least 95% by weight and more particularly 99% by weight of the B form along with the other polymorphic forms.

The B form of ethyl [(7S)-7(2R)-2-(3-chlorophenyl)-2-hydroxyethylamino-5,6,7,8-tetrahydronaphthalen-2-yloxy]-acetate hydrochloride is another example of a phenyl-ethanolaminotetralin that may be used in the context of the invention.

According to another embodiment of the invention, the β3 adrenergic receptor agonist is chosen from arylethanol-diamines in the form of base or of acid-addition salt, such as those described in document WO 02/066 418, for example 3′-[[2-[[(2R)-2-(3-chlorophenyl)-2-hydroxyethyl]amino]-ethyl]amino]biphenyl-3-carboxylic acid or the hydrochloride salt thereof.

The combination according to the invention also comprises an MARI, in base or acid-addition salt form.

The MARI may be chosen especially from fluoxetine, fluoxetine hydrochloride, citalopram, citalopram hydrobromide, fluvoxamine, fluvoxamine maleate, paroxetine, paroxetine hydrochloride, sertraline, sertraline hydrochloride, milnacipran, milnacipran hydrochloride, escitalopram (S-citalopram), escitalopram oxalate, duloxetine, duloxetine hydrochloride, venlafaxine, venlafaxine hydrochloride, desvenlafaxine, radafaxin, bupropion and bupropion hydrochloride, and also mixtures thereof.
Fluoxetin or N-methyl-3-(p-trifluoromethoxyphenoxy)-3-phenylpropalamine is sold in hydrochloride form and as a racemic mixture of the two R and S enantiomers.

For the purposes of the present invention, the term “fluoxetine” means the compound in free base or acid-addition salt form, also including the racemic mixture or the R or S enantiomers alone.
Duloxetine or N-methyl-3-(1-naphthalenyloxy)-3-(2-thienyl)propanamine is also known and is generally administered in the form of the hydrochloride of the (+) enantiomer.
Venlafaxine is described in the literature. Its synthetic process and its activity as a serotonin and norepinephrine reuptake inhibitor are described in document U.S. Pat. No. 4,761,501.
Milnacipran or N,N-diethyl-2-aminomethyl-1-phenyl-cyclopropanecarboxamide is described in document U.S. Pat. No. 4,478,836. The pharmacological activity of milnacipran as a serotonin and noradrenalin reuptake inhibitor is described by Moret et al., Neuropharmacology 24, 1211-19 (1985).
Citalopram or 1-[(3-(dimethylamino)propyl]-1-(4-fluoro-phenyl)-1,3-dihydro-5-isobenzofurancarbonitrile is described in document U.S. Pat. No. 4,136,193.
Escitalopram (S-citalopram) or (+)-1-[3-(dimethyl-amino)propyl]-1-(4-fluorophenyl)-1,3-dihydro-5-isobenzofurancarbonitrile is described in document EP 0 347 066.
Fluvoxamine or 5-methoxy-1-[4-(trifluoromethyl)-phenyl]-1-pentanone O-(2-aminoethyl)oxime is described in document U.S. Pat. No. 4,085,225.
Paroxetine or trans-(−)-3-[(1,3-benzodioxol-5-yloxy)methyl]-4-(4-fluorophenyl)piperidine is described in documents U.S. Pat. No. 3,912,743 and U.S. Pat. No. 4,007,196.
Sertraline, (1S-cis)-4-(3,4-dichlorophenyl)-1,2,3,4-tetrahydro-N-methyl-1-naphthylamine hydrochloride, is a serotonin reuptake inhibitor, sold as an antidepressant. This compound is described in document U.S. Pat. No. 4,536,518.
Desvenlafaxine or 4-[(1RS)-2-(dimethylamino)-(1-hydroxycyclohexyl)ethyl]phenol and also radafaxin are described in the literature.
Bupropion or 1-(3-chlorophenyl)-2-[(1,1-dimethyl-ethyl)amino]-1-propanone is described in document U.S. Pat. No. 3,819,706.

According to one embodiment variant, MARIs that may be used according to the invention are serotonin reuptake inhibitors, for example fluoxetine and escitalopram.

Thus, a first example of a combination according to the invention is the combination consisting of ethyl [(7S)-7(2R)-2-(3-chlorophenyl)-2-hydroxyethylamino-5,6,7,8-tetra-hydronaphthalen-2-yloxy]acetate hydrochloride and of fluoxetine, for example fluoxetine hydrochloride.

Another example of a combination according to the invention is the combination consisting of ethyl [(7S)-7(2R)-2-(3-chlorophenyl)-2-hydroxyethylamino-5,6,7,8-tetrahydro-naphthalen-2-yloxy]acetate hydrochloride and of escitalopram, for example escitalopram oxalate.

A second subject of the invention relates to a pharmaceutical composition comprising, as active principle, a combination as defined above, and one or more pharmaceutically acceptable excipients.

The pharmaceutical composition contains an effective dose of each active principle present in the combination according to the invention.

The excipients are chosen, according to the desired pharmaceutical form and the desired mode of administration, from the usual excipients known to those skilled in the art.

The composition may be administered orally, parenterally or rectally.

The appropriate unit forms of administration include oral forms such as tablets, soft or hard gel capsules, powders, granules and oral solutions or suspensions, sublingual, buccal, in tracheal, intraocular and intranasal administration forms, forms for inhalation, topical, transdermal, subcutaneous, intramuscular or intravenous administration forms, rectal administration forms and implants. For topical application, the active principles according to the invention may be used in creams, gels, pomades or lotions.

According to the invention, the two active principles are administered according to the same route, for example the oral route, or one of the active principles is administered according to a first route, for example the oral route, and the other active principle is administered according to a different route, for example the parenteral route.

When a composition is prepared in tablet form, the active ingredients are mixed with one or more pharmaceutical excipients, such as gelatine, starch, lactose, magnesium stearate, talc, silica, gum arabic, mannitol, microcrystalline cellulose, hypromellose or analogues.

The tablets may be coated with sucrose, with a cellulose derivative or with other materials suitable for coating. The tablets may be made via various techniques, such as direct compression, wet or dry granulation or hot melting.

A pharmaceutical composition may also be obtained in gel capsule form by mixing the active ingredients with a diluent and pouring the mixture obtained into soft or hard gel capsules.

Aqueous suspensions, isotonic saline solutions or sterile injectable solutions containing pharmacologically compatible agents, for example propylene glycol or butylene glycol, are used for parenteral administration.

According to the usual practice, the dosage that is suitable for each patient is determined by the doctor according to the mode of administration, the age, the weight and the response of the said patient.

The doses depend on the desired effect, the duration of the treatment and the administration route used.

For example, via the oral route, the daily doses of each of the active principles of the combination according to the invention are as follows:
β3 receptor agonist: between 10 and 2000 mg per day per person, especially between 50 and 1000 mg per day per person;
MARI: between 5 and 800 mg per day per person, especially between 10 and 500 mg per day per person.

There may be special cases where higher or lower dosages are suitable. Such dosages do not depart from the context of the invention.

The respective doses of the β3 agonist and of MARI are generally substantially identical to each other or may differ from each other.

By way of example, a unit form of administration of the β3 receptor agonist in tablet form comprises the following ingredients:

| | |
|---|---|
| Ethyl [(7S)-7(2R)- | 50 mg |
| 2-(3-chlorophenyl)- | |
| 2-hydroxyethylamino-5,6,7,8- | |
| tetrahydronaphthalen-2-yloxy] | |
| acetate hydrochloride | |
| Mannitol | 174 mg |
| Sodium croscarmellose | 6 mg |
| Corn starch | 15 mg |
| Hydroxypropylmethylcellulose | 2 mg |
| Magnesium stearate | 3 mg |

Also as an example, a unit form of administration of escitalopram in tablet form may comprise 10 mg of escitalopram oxalate and common excipients, for example microcrystalline cellulose, colloidal silica, talc, sodium croscarmellose or magnesium stearate.

The administration of each of the active principles may also be performed simultaneously, separately or sequentially over time (sequential administration).

When the administration is performed simultaneously, the two active principles may be combined in a single pharmaceutical composition, comprising the two active principles, such as a tablet or a gel capsule.

The two active principles may also, whether or not they are administered simultaneously, be present in separate pharmaceutical compositions. To this end, the combination according to the invention may be in the form of a kit comprising, on the one hand, at least one β3 agonist as defined hereinabove and, on the other hand, at least one MARI as defined hereinabove, the β3 receptor agonist and the MARI being in separate compartments and being intended to be administered simultaneously, separately or sequentially over time (sequential administration).

Another subject of the invention relates to the use of a combination as described above for the preparation of a medicament for treating and/or preventing depression or anxiety.

A subject of the invention is also a method for treating depression or anxiety, which comprises the administration to a patient of a therapeutically effective dose of at least one β3 agonist as defined above, and of at least one therapeutically effective dose of at least one MARI as defined hereinabove, the said doses being administered simultaneously, separately or sequentially, as described previously.

The antidepressant effects of the combination according to the invention were evaluated in mice using the moderate chronic stress (MCS) protocol. This test was developed on rats by Willner (P. Willner, Neuroscience and Biobehavioral Review, 1992, 16, 525-34) and adapted to mice by Kopp et al. (C. Kopp, Behavioral Pharmacology, 1999, 10, 73-83).

Similarities between the depressive state in man and the effect of moderate chronic stress in mice were established. The emotivity of mice stressed by MCS was evaluated in the forced swimming test.

The MCS is performed in the following manner:

Male BALB/c ByJIco mice (Iffa Credo Lyons, France) 8 weeks old are placed in individual experiment cages (26×20×14 cm), provided with food and water ad libitum, at a controlled temperature of 22±1° C.

In the context of the protocol, the mice are subjected to a series of stresses such as forced bathing, food and/or water deprivation, housing in cages containing wet sawdust, permutation of the day/night cycle, maintenance under constant illumination or in darkness, each of these restrictions possibly lasting from 2 hours to 24 hours.

A first group of control animals is not subjected to the MCS (group 1—unstressed).

The animals subjected to the MCS are divided into 4 groups on day 15:
Group 2 (saline stress) consists of control animals receiving one intraperitoneal injection per day of physiological fluid.
Group 3 (fluoxetine stress) receives 1 intraperitoneal injection per day of fluoxetine hydrochloride at a rate of 3 mg/kg.
Group 4 (β3 agonist stress) receives 1 intraperitoneal injection per day of ethyl [(7S)-7(2R)-2-(3-chlorophenyl)-2-hydroxyethylamino-5,6,7,8-tetrahydronaphthalen-2-yloxy]acetate hydrochloride at a rate of 1 mg/kg.
Group 5 (fluoxetine+β3 agonist stress) receives 1 intraperitoneal injection per day of a solution containing fluoxetine hydrochloride (3 mg/kg) and ethyl [(7S)-7(2R)-2-(3-chlorophenyl)-2-hydroxyethylamino-5,6,7,8-tetrahydro-naphthalen-2-yloxy]acetate hydrochloride (1 mg/kg).

The injections are performed from day 15 to day 50.

### Emotivity During the Forced Swimming Test

On day 50, each group is subjected to the forced swimming test. This test consists in bathing each animal in a glass cylinder containing water and in evaluating the degree of adaptation exhibited by the mouse to the stress. The immobile time is measured over a period of 5 minutes.

The results are given in Table 1 below.

TABLE 1

| Group | Immobile time (seconds) |
|---|---|
| 1 (unstressed) | 146.8 |
| 2 (saline stress) | 193.9 |
| 3 (fluoxetine stress) | 178.4 |
| 4 (β3 agonist stress) | 182.0 |
| 5 (fluoxetine + β3 agonist stress) | 153.6 |

The percentage of immobile time for the groups of stressed animals relative to the unstressed animals is also calculated and indicated in Table 2 below.

TABLE 2

| Group | Percentage of time |
|---|---|
| 1 (unstressed) | — |
| 2 (saline stress) | +32.1% |
| 3 (fluoxetine stress) | +21.5% |
| 4 (β3 agonist stress) | +25.1% |
| 5 (fluoxetine + β3 agonist stress) | +4% |

These results show an increase in the immobile time of the stressed animals compared with the unstressed animals after exposure to the MCS. The repeated administration of β3 agonist alone or of fluoxetine alone does not significantly modify this parameter.

Moreover, the repeated administration of the combination of fluoxetine and ethyl [(7S)-7(2R)-2-(3-chlorophenyl)-2-hydroxyethylamino-5,6,7,8-tetrahydronaphthalen-2-yloxy]-acetate produces a pronounced reduction of the immobile time compared with the other groups of stressed animals (groups 2 to 4).

Table 2 also shows that the performance of the mice treated repeatedly with the combination of fluoxetine and ethyl [(7S)-7(2R)-2-(3-chlorophenyl)-2-hydroxyethylamino-5,6,7,8-tetrahydronaphthalen-2-yloxy]acetate is of the same level as the performance of the unstressed mice.

The above experiments thus show that the invention produces a synergistic effect of antidepressant activity that is higher in terms of efficacy than that of each of the active principles administered individually at the same dosage.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A combination comprising at least one ingredient selected from the group consisting of β3 adrenergic receptor agonist chosen from arylethanoldiamines and pharmaceutically acceptable salts thereof and phenylethanolaminotetralins of formula (I): 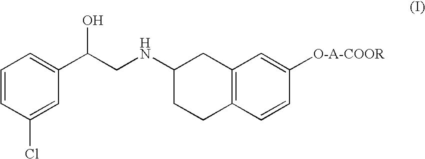
wherein:
A represents a C₁₋₄ alkylene group, and
R represents a hydrogen atom or a C₁₋₄ alkyl group, and pharmaceutically acceptable salts thereof, and hydrates and solvates thereof, and
at least one second active ingredient chosen from monoamine reuptake inhibitor (MARI) and pharmaceutically acceptable salts thereof.

2. The combination according to claim 1, wherein the arylethanoldiamines is 3′-[[2-[[(2R)-2-(3-chlorophenyl)-2-hydroxyethyl]amino]ethyl]amino]biphenyl-3-carboxylic acid or a hydrochloride salt thereof.

3. The combination according to claim 1, wherein the phenylethanolaminotetralin is of the formula (I): 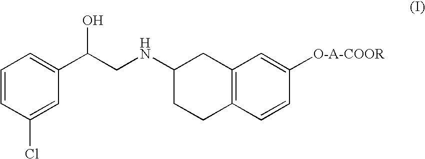
wherein:
A represents a methylene or isopropylidene group, and
R represents a hydrogen atom or a C₁₋₄ alkyl group.

4. The combination according to claim 1, wherein the phenylethanolaminotetralin is ethyl [2-(3-chlorophenyl)-2-hydroxyethylamino-5,6,7,8-tetrahydro-naphthalen-2-yloxy]acetate.

5. The combination according to claim 1, wherein the phenylethanolaminotetralin is ethyl [2-(3-chlorophenyl)-2-hydroxyethylamino-5,6,7,8-tetrahydronaphthalen-2-yloxy]acetate hydrochloride.

6. The combination according to claim 1, wherein the phenylethanolaminotetralin is ethyl [(7S)-7(2R)-2-(3-chlorophenyl)-2-hydroxyethylamino-5,6,7,8-tetrahydronaphthalen-2-yloxy]acetate.

7. The combination according to claim 1, wherein the phenylethanolaminotetralin is ethyl [(7S)-7(2R)-2-(3-chlorophenyl)-2-hydroxyethylamino-5,6,7,8-tetrahydronaphthalen-2-yloxy]acetate hydrochloride.

8. The combination according to claim 1, wherein the phenylethanolaminotetralin is ethyl [(7S)-7(2R)-2-(3-chlorophenyl)-2-hydroxyethylamino-5,6,7,8-tetrahydronaphthalen-2-yloxy]acetate hydrochloride in B form, the infrared spectrum of which shows the following characteristic absorption peaks: 2780, 2736, 1722, 1211 cm⁻¹.

9. The combination according to claim 1, wherein the MARI is selected from the group consisting of: fluoxetine, fluoxetine hydrochloride, citalopram, citalopram hydrobromide, fluvoxamine, fluvoxamine maleate, paroxetine, paroxetine hydrochloride, sertraline, sertraline hydrochloride, milnacipran, milnacipran hydrochloride, escitalopram (S-citalopram), escitalopram oxalate, duloxetine, duloxetine hydrochloride, venlafaxine, venlafaxine hydrochloride, desvenlafaxine, radafaxine, bupropion and bupropion hydrochloride, and mixtures in any combination thereof.

10. The combination according to claim 9, wherein the MARI is selected from the group consisting of: fluoxetine, fluoxetine hydrochloride, escitalopram, and escitalopram oxalate.

11. The combination according to claim 1, wherein the β3 agonist is ethyl [(7S)-7(2R)-2-(3-chlorophenyl)-2-hydroxyethylamino-5,6,7,8-tetrahydronaphthalen-2-yloxy]acetate hydrochloride and the MARI is fluoxetine, or fluoxetine hydrochloride.

12. The combination according to claim 1, wherein the β3 agonist is ethyl [(7S)-7(2R)-2-(3-chlorophenyl)-2-hydroxyethylamino-5,6,7,8-tetrahydronaphthalen-2-yloxy]acetate hydrochloride and the MARI is escitalopram, or escitalopram oxalate.

13. A pharmaceutical composition comprising at least one β3 adrenergic receptor agonist chosen from phenylethanolaminotetralins of formula (I): 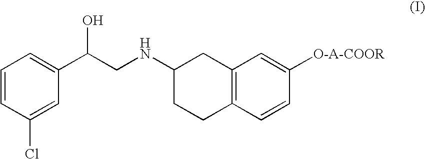
wherein:
A represents a C₁₋₄ alkylene group, and
R represents a hydrogen atom or a C₁₋₄ alkyl group, or a pharmaceutically acceptable salt thereof, or a hydrate or a solvate thereof, and
at least one monoamine reuptake inhibitor (MARI), in combination with at least one pharmaceutically acceptable excipient.

14. The pharmaceutical composition according to claim 13, wherein the phenylethanolaminotetralin corresponds to the formula (I) in which:
A represents a methylene or isopropylidene group, and
R represents a hydrogen atom or a C₁₋₄ alkyl group.

15. The pharmaceutical composition according to claim 12, wherein the phenylethanolaminotetralin is selected from the group consisting of:
ethyl [2-(3-chlorophenyl)-2-hydroxyethylamino-5,6,7,8-tetrahydronaphthalen-2-yloxy]acetate;
ethyl [(7S)-7(2R)-2-(3-chlorophenyl)-2-hydroxyethylamino-5,6,7,8-tetrahydronaphthalen-2-yloxy]acetate;
ethyl [2-(3-chlorophenyl)-2-hydroxyethylamino-5,6,7,8-tetrahydronaphthalen-2-yloxy]acetate hydrochloride;
ethyl [(7S)-7(2R)-2-(3-chlorophenyl)-2-hydroxy-ethylamino-5,6,7,8-tetrahydronaphthalen-2-yloxy]acetate hydrochloride; and
ethyl [(7S)-7(2R)-2-(3-chlorophenyl)-2-hydroxyethyl-amino-5,6,7,8-tetrahydronaphthalen-2-yloxy]acetate hydrochloride in B form.

16. The pharmaceutical composition according to claim 13, wherein the MARI is selected from the group consisting of: fluoxetine, fluoxetine hydrochloride, citalopram, citalopram hydrobromide, fluvoxamine, fluvoxamine maleate, paroxetine, paroxetine hydrochloride, sertraline, sertraline hydrochloride, milnacipran, milnacipran hydrochloride, escitalopram (S-citalopram), escitalopram oxalate, duloxetine, duloxetine hydrochloride, venlafaxine, venlafaxine hydrochloride, desvenlafaxine, radafaxine, bupropion and bupropion hydrochloride, and mixtures in any combination thereof.

17. The pharmaceutical composition according to claim 16, wherein the MARI is fluoxetine or escitalopram.

18. The pharmaceutical composition according to claim 13, wherein said β3 adrenergic receptor agonist and said MARI are administered simultaneously, separately or sequentially over time.

19. A method of treatment of depression or anxiety in a patient comprising administering to the patient a therapeutically effective amount of a combination of at least one β3 adrenergic receptor agonist chosen from arylethanoldiamines and pharmaceutically acceptable salts thereof, and phenylethanolaminotetralins of formula (I): 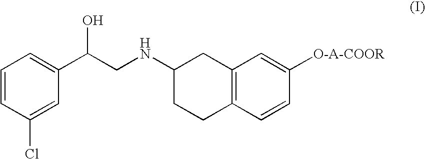
wherein:
A represents a C₁₋₄ alkylene group, and
R represents a hydrogen atom or a C₁₋₄ alkyl group, and pharmaceutically acceptable salts thereof, and hydrates or solvates thereof, and
at least one monoamine reuptake inhibitor (MARI).

20. The method according to claim 19, wherein the arylethanoldiamine is 3′-[[2-[[(2R)-2-(3-chlorophenyl)-2-hydroxyethyl]amino]ethyl]-amino]-biphenyl-3-carboxylic acid or the hydrochloride salt thereof.

21. The method according to claim 19, wherein the phenylethanolaminotetralin is selected from the group consisting of:
ethyl [2-(3-chlorophenyl)-2-hydroxyethylamino-5,6,7,8-tetrahydronaphthalen-2-yloxy]acetate;
ethyl [(7S)-7(2R)-2-(3-chlorophenyl)-2-hydroxyethylamino-5,6,7,8-tetrahydronaphthalen-2-yloxy]acetate;
ethyl [2-(3-chlorophenyl)-2-hydroxyethylamino-5,6,7,8-tetrahydronaphthalen-2-yloxy]acetate hydrochloride;
ethyl [(7S)-7(2R)-2-(3-chlorophenyl)-2-hydroxy-ethylamino-5,6,7,8-tetrahydronaphthalen-2-yloxy]acetate hydrochloride; and
ethyl [(7S)-7(2R)-2-(3-chlorophenyl)-2-hydroxyethyl-amino-5,6,7,8-tetrahydronaphthalen-2-yloxy]acetate hydrochloride in B form.

22. The method according to claim 19, wherein the MARI is selected from the group consisting of: fluoxetine, fluoxetine hydrochloride, citalopram, citalopram hydrobromide, fluvoxamine, fluvoxamine maleate, paroxetine, paroxetine hydrochloride, sertraline, sertraline hydrochloride, milnacipran, milnacipran hydrochloride, escitalopram (S-citalopram), escitalopram oxalate, duloxetine, duloxetine hydrochloride, venlafaxine, venlafaxine hydrochloride, desvenlafaxine, radafaxine, bupropion and bupropion hydrochloride, and mixtures in any combination thereof.

23. The method according to claim 22, wherein the MARI is fluoxetine or escitalopram.

24. The method according to claim 19, wherein said β3 adrenergic receptor agonist and said MARI are administered simultaneously, separately or sequentially over time.

25. The method according to claim 19, wherein the disease is depression.

26. The method according to claim 19, wherein the disease is anxiety.
